(19) United States
(12) Patent Application Publication
(10) Pub. No.: US 2022/0242843 A1
(43) Pub. Date: Aug. 4, 2022

(54) PROCESS FOR PREPARING A COUMARIN-CAGED FORSKOLIN DERIVATIVE, FORSKOLIN DERIVATIVE AND USE OF SAID FORSKOLIN DERIVATIVE

(71) Applicant: Forschungszentrum Juelich GmbH, Juelich (DE)

(72) Inventors: Arnd Baumann, Juelich (DE); Dirk Bier, Bottrop (DE); Marcus Holschbach, Aachen (DE); Birte Drewes, Juelich (DE); Thomas Gensch, Aachen (DE); Bernd Neumaier, Cologne (DE); Sabine Balfanz, Juelich (DE)

(21) Appl. No.: 17/622,267

(22) PCT Filed: Jul. 2, 2020
(86) PCT No.: PCT/DE2020/000146

§ 371 Date: Dec. 23, 2021

(30) Foreign Application Priority Data
Jul. 25, 2019 (DE) 10 2019 005 196.3

(51) Int. Cl. C07D 311/18; C07D 311/78; C07D 311/92; C07D 493/08; C07D 407/12; C09K 11/06; G01N 21/64

(52) U.S. Cl. CPC C07D 311/18; C07D 311/78; C07D 311/92; G01N 21/6428; C07D 407/12; C09K 11/06; C07D 493/08

(57) ABSTRACT

A process for preparing coumarin-caged forskolin derivatives, the forskolin derivatives themselves and to uses of the same.

### CROSS-REFERENCE TO PRIOR APPLICATIONS

This application is a U.S. National Phase application under 35 U.S.C. § 371 of International Application No. PCT/DE2020/000146, filed on Jul. 2, 2020, and claims benefit to German Patent Application No. DE 10 2019 005 196.3, filed on Jul. 25, 2019. The International Application was published in German on Jan. 28, 2021 as WO 2021/013278 A1 under PCT Article 21(2).

### FIELD

Embodiments of the invention relate to a process for preparing a coumarin-caged forskolin derivative, to a corresponding forskolin derivative, and to the use of the forskolin derivative.

### BACKGROUND

Forskolin is a diterpene of the labdane type from Coleus forskohlii (syn. Plectranthus barbatus) from the family of the Lamiaceae.

It is known that forskolin activates adenylate cyclases directly but non-specifically. It also leads to an increase in the intracellular cAMP concentration. Various cAMP-dependent signal transduction pathways can be influenced via this approach. Forskolin is therefore used as a tool in experimental pharmacology (König, Gabriele M. Forskolin. last update: May 2012. In: Römpp [online]: Georg Thieme Verlag KG [retrieved on: Jun. 17, 2019] available at: roempp.thieme.de.

What are known as caged compounds are used for this purpose, among other things. Caged compounds are chemically modified compounds that release a defined substance upon irradiation with light of certain wavelengths. Their main field of application is biochemical and cytological research.^{1,2} Biologically active compounds are provided with a photolabile protecting group (“cage”) and are thus temporarily biologically inactive. By means of light irradiation, the photolabile protecting group is irreversibly cleaved, and the previously inactive compound again has its specific biological activity. Caged compounds are used to release effectors at a specific location at a specific time if their direct application is difficult or too slow to achieve the desired concentration directly at the site of action, such as inside a cell. In contrast, the inactive caged compound can also accumulate at the target by slow diffusion and, upon subsequent exposure to light, release a sufficient quantity of effector in a short period of time.

By using suitable flash lamps or lasers, it is possible to start a biochemical process, e.g., an enzymatically catalyzed reaction or signal transmission very quickly (picoseconds to milliseconds).

Caged compounds could be used to investigate the time-dependence of G-protein-coupled receptor (GPCR)-mediated signaling cascades, in the present case to conduct a detailed examination of an adenylyl cyclase-mediated signaling pathway.

Coumarin is an established cage group, previously, e.g., of caged mRNA and DNA.³ G-protein-coupled receptors (GPCRs) form the largest family of membrane-bound receptors and regulate a large number of cellular processes. The activation of GPCRs induces intracellular changes in the concentration of secondary messenger substances such as cyclic adenosine-3′,5′-monophosphate (cAMP) and calcium (Ca²⁺). These secondary effects are induced by specific signaling cascades. The activation of membrane-bound adenylate cyclases (ACs) leads to the production of cAMP.

The naturally occurring diterpene forskolin is used experimentally in biochemistry and pharmacology as a direct stimulator of adenylate cyclase.^{4,5} The conversion of adenosine triphosphate (ATP) to the signaling substance cAMP is catalyzed in the cell as a result of enzyme activation. In this way, forskolin intervenes centrally in the signal transduction pathways of many G-protein-coupled receptors.

Forskolin is thus used experimentally as a direct stimulator of adenylyl cyclases (ACs). Water-soluble forskolin derivatives, such as the commercially available Colforsin (NKH 477, see FIG. 1),⁶ are typically acylated with a polar aliphatic amine at C-6 or C-7.^{7,8} These derivatives are generally even more selective for adenylyl cyclases and have lower off-target activities.⁹

Disadvantageously, however, there are no water-soluble forskolin derivatives that can be released in a light-controlled manner. There is thus no “inactive” caged compound of the forskolin, which accumulates at the target by diffusion and can be intentionally released directly at the site of action in a very short time by subsequent photolysis. A primary difficulty is the complexity of a synthesis route.

### SUMMARY

According to an embodiment of the present invention, a process is provided for preparing a specific coumarin-caged forskolin derivative JCF 1 of formula

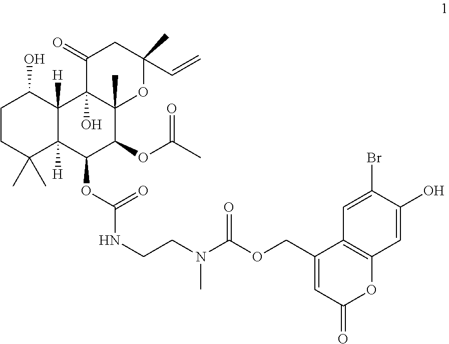

is characterized by the steps of:

a. synthesizing ((6-bromo-7-(methoxymethoxy)-2-oxo-2H-chromen-4-yl)methyl methyl(2-(2,2,2-trifluoroacetamido)ethyl)carbamate 4 of formula

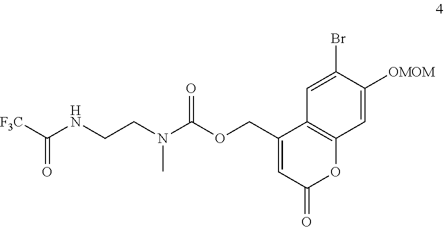

by a first carbamoylation of 2,2,2-trifluoro-N-(2-methylamino-ethyl)-acetamide 5 and 6-bromo-7-methoxymethoxy coumarin-4-ylmethyl 4′-nitrophenyl carbonate 6 (FIG. 3),
b. synthesizing (6-bromo-7-(methoxymethoxy)-2-oxo-2H-chromen-4-yl)methyl (2-aminoethyl)(methyl)carbamate 3 of formula

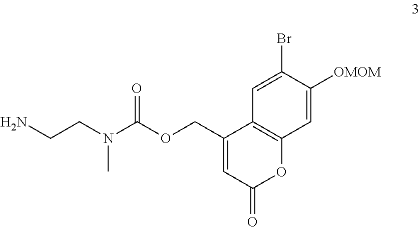

by a first deprotection of (6-bromo-7-(methoxymethoxy)-2-oxo-2H-chromen-4-yl)methyl methyl(2-(2,2,2-trifluoroacetamido)ethyl)carbamate 4 (FIG. 4),
c. synthesizing (6-bromo-7-(methoxymethoxy)-2-oxo-2H-chromen-4-yl)methyl (2-(((((2R,4aR,4a1R,6S,10aS,11S,12S,12aR)-6-(dimethylamino)-12-hydroxy-2,4a1,10,10,12a-pentamethyl-4-oxo-2-vinyldecahydro-2H,8H-pyrano[3′,2′:1,2]naphtho[1,8-de][1,3]dioxin-11-yl)oxy)carbonyl)amino)ethyl)(methyl)carbamate 7 according to the formula

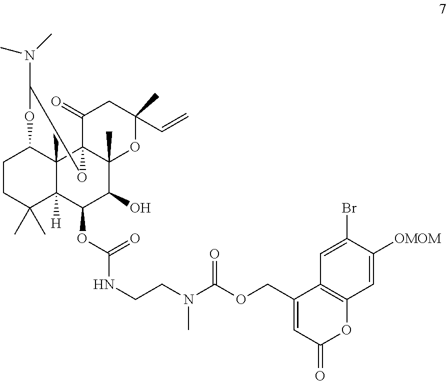

by a second carbamoylation of 7-deacetyl forskolin-6,7-carbonate 1,9-dimethylformamide dimethyl acetal 2 and (6-bromo-7-(methoxymethoxy)-2-oxo-2H-chromen-4-yl)methyl (2-aminoethyl)(methyl)carbamate 3 (FIG. 5),
d. synthesizing (2R,4aR,4a1R,6S,10aS,11S,12S,12aR)-11-(((2-((((6-bromo-7-(methoxymethoxy)-2-oxo-2H-chromen-4-yl)methoxy)carbonyl)(methyl)amino)ethyl)carbamoyl)oxy)-6-(dimethylamino)-2,4a1,10,10,12a-pentamethyl-4-oxo-2-vinyldecahydro-2H,8H-pyrano[3′,2′:1,2]naphtho[1,8-de][1,3]dioxin-12-yl acetate 8 according to the formula

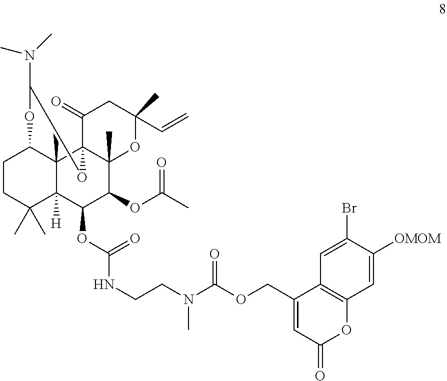

by acetylation of (6-bromo-7-(methoxymethoxy)-2-oxo-2H-chromen-4-yl)methyl (2-(((((2R,4aR,4a1R,6S,10aS,11S,12S,12aR)-6-(dimethylamino)-12-hydroxy-2,4a1,10,10,12a-pentamethyl-4-oxo-2-vinyldecahydro-2H,8H-pyrano[3′,2′:1,2]naphtho[1,8-de][1,3]dioxin-11-yl)oxy)carbonyl)amino)ethyl)(methyl)carbamate 7 (FIG. 6),
e. synthesizing (3R,4aR,5S,6S,6aS,10S,10aR,10bS)-6-(((2-((((6-bromo-7-(methoxymethoxy)-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-yl acetate 9 according to the formula

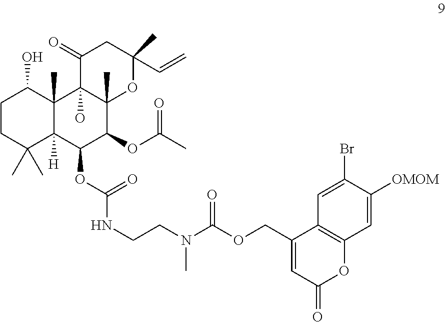

by a second deprotection of (2R,4aR,4a1R,6S,10aS,11S,12S,12aR)-11-(((2-((((6-bromo-7-(methoxymethoxy)-2-oxo-2H-chromen-4-yl)methoxy)carbonyl)(methyl)amino)ethyl)carbamoyl)oxy)-6-(dimethylamino)-2,4a1,10,10,12a-pentamethyl-4-oxo-2-vinyldecahydro-2H,8H-pyrano[3′,2′:1,2]naphtho[1,8-de][1,3]dioxin-12-yl acetate 8 (FIG. 7),
f. synthesizing (3R,4aR,5S,6S,6aS,10S,10aR,10bS)-6-(((2-((((6-bromo-7-hydroxy-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-yl acetate (JCF 1) according to the formula

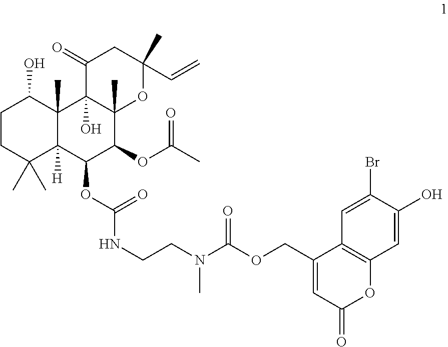

by a third deprotection of (3R,4aR,5S,6S,6aS,10S,10aR,10bS)-6-(((2-((((6-bromo-7-(methoxymethoxy)-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-yl acetate 9 (FIG. 8).
In further embodiments, intermediates of the processes disclosed herein are provided, including those of formulas 3, 4, and 7-9.
In still further embodiments, methods for the use of the products of the processes disclosed herein are provided.

### BRIEF DESCRIPTION OF THE DRAWINGS

FIG. 1: Forskolin and NKH 477.

FIG. 2: Synthesis overview of JCF 1 consisting of protected forskolin 2 and a coumarin derivative 3 functionalized with N-methylethylenediamine.

FIG. 3: Synthesis of (6-bromo-7-(methoxymethoxy)-2-oxo-2H-chromen-4-yl)methyl methyl(2-(2,2,2-trifluoroacetamido)ethyl)carbamate 4 (step a.).

FIG. 4: Synthesis of (6-bromo-7-(methoxymethoxy)-2-oxo-2H-chromen-4-yl)methyl(2-aminoethyl)(methyl)carbamate 3 (step b.).

FIG. 5: Synthesis of (6-bromo-7-(methoxymethoxy)-2-oxo-2H-chromen-4-yl)methyl(2-(((((2R,4aR,4a1R,6S,10aS,11S,12S,12aR)-6-(dimethylamino)-12-hydroxy-2,4a1,10,10,12a-pentamethyl-4-oxo-2-vinyldecahydro-2H,8H-pyrano[3′,2′:1,2]naphtho[1,8-de][1,3]dioxin-11-yl)oxy)carbonyl)amino)ethyl)(methyl)carbamate 7 (step c.).

FIG. 6: Synthesis of (2R,4aR,4a1R,6S,10aS,11S,12S,12aR)-11-(((2-((((6-bromo-7-(methoxymethoxy)-2-oxo-2H-chromen-4-yl)methoxy)carbonyl)(methyl)amino)ethyl)carbamoyl)oxy)-6-(dimethylamino)-2,4a1,10,10,12a-pentamethyl-4-oxo-2-vinyldecahydro-2H,8H-pyrano[3′,2′:1,2]naphtho[1,8-de][1,3]dioxin-12-ylacetate 8 (step d.).

FIG. 7: Synthesis of (3R,4aR,5S,6S,6aS,10S,10aR,10bS)-6-(((2-((((6-bromo-7-(methoxymethoxy)-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-yl acetate 9 (step e.).

FIG. 8: Synthesis of (3R,4aR,5S,6S,6aS,10S,10aR,10bS)-6-(((2-((((6-bromo-7-hydroxy-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-yl acetate, (JCF 1) (step f.).

FIG. 9: Photolysis of JCF 1

FIG. 10: Irradiation of JCF 1 and cleavage to form forskolin carbamate 10

FIG. 11: Relative fluorescence when loaded with 10 μM JCF 1; the time points were measured at an interval of 1 min.

FIG. 12: Relative fluorescence when loaded with 30 μM JCF 1; the time points were measured at an interval of 1 min.

FIG. 13: Relative fluorescence when loaded with 10 μM NHK 477; the time points were measured at an interval of 1 min.

FIG. 14: Relative fluorescence when loaded with 30 μM NHK 477; the time points were measured at an interval of 1 min.

FIG. 15: Relative fluorescence without loading (negative control); the time points were measured at an interval of 1 min.

### DETAILED DESCRIPTION

In an embodiment, the present invention provides a process for preparing “coumarin-caged forskolin derivatives”. Furthermore, the present invention provides in another embodiment the corresponding coumarin-caged forskolin derivatives in order to be able to conduct cell- or tissue-based investigations.

According to an embodiment of the present invention, a process is provided for preparing a specific coumarin-caged forskolin derivative JCF 1 of formula

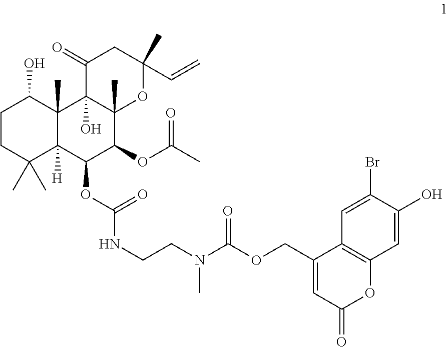

is characterized by the steps of:

a. synthesizing ((6-bromo-7-(methoxymethoxy)-2-oxo-2H-chromen-4-yl)methyl methyl(2-(2,2,2-trifluoroacetamido)ethyl)carbamate 4 of formula

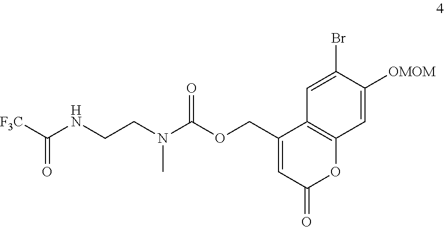

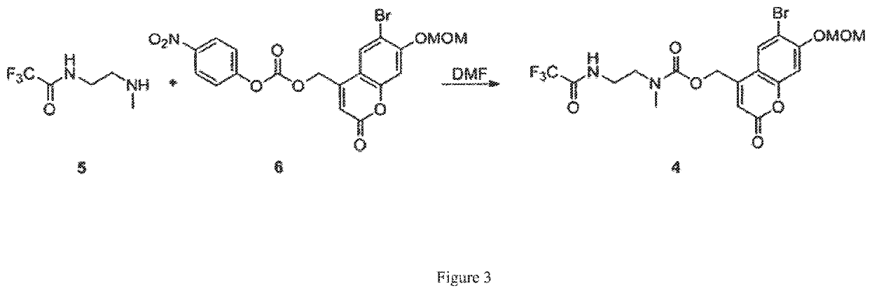

by a first carbamoylation of 2,2,2-trifluoro-N-(2-methylamino-ethyl)-acetamide 5 and 6-bromo-7-methoxymethoxy coumarin-4-ylmethyl 4′-nitrophenyl carbonate 6 (FIG. 3),
b. synthesizing (6-bromo-7-(methoxymethoxy)-2-oxo-2H-chromen-4-yl)methyl (2-aminoethyl)(methyl)carbamate 3 of formula

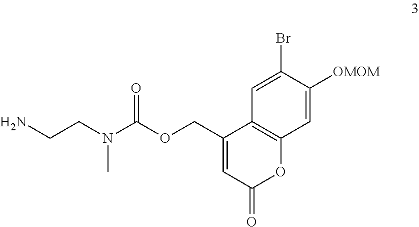

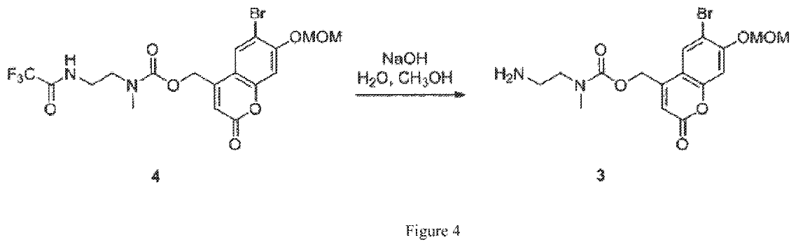

by a first deprotection of (6-bromo-7-(methoxymethoxy)-2-oxo-2H-chromen-4-yl)methyl methyl(2-(2,2,2-trifluoroacetamido)ethyl)carbamate 4 (FIG. 4),
c. synthesizing (6-bromo-7-(methoxymethoxy)-2-oxo-2H-chromen-4-yl)methyl (2-(((((2R,4aR,4a1R,6S,10aS,11S,12S,12aR)-6-(dimethylamino)-12-hydroxy-2,4a1,10,10,12a-pentamethyl-4-oxo-2-vinyldecahydro-2H,8H-pyrano[3′,2′:1,2]naphtho[1,8-de][1,3]dioxin-11-yl)oxy)carbonyl)amino)ethyl)(methyl)carbamate 7 according to the formula

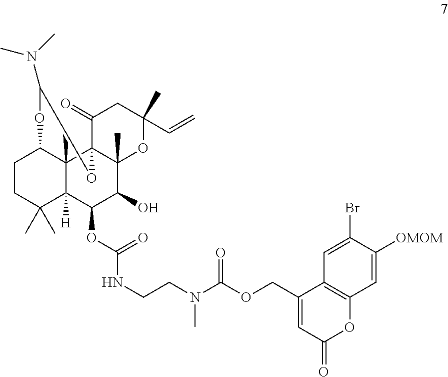

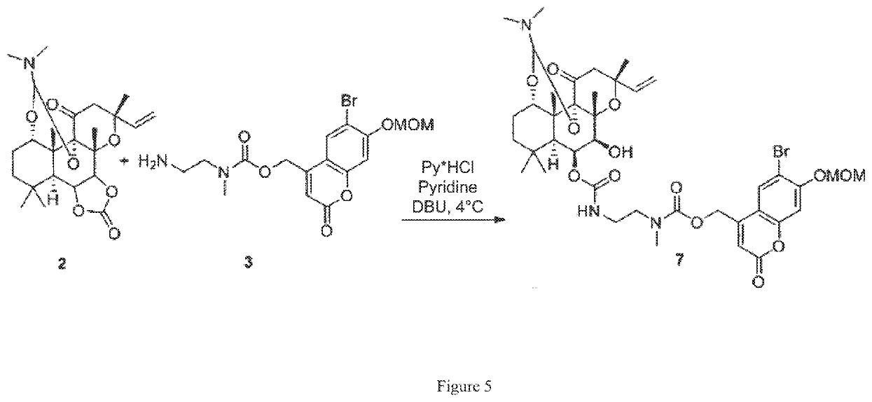

by a second carbamoylation of 7-deacetyl forskolin-6,7-carbonate 1,9-dimethylformamide dimethyl acetal 2 and (6-bromo-7-(methoxymethoxy)-2-oxo-2H-chromen-4-yl)methyl (2-aminoethyl)(methyl)carbamate 3 (FIG. 5),
d. synthesizing (2R,4aR,4a1R,6S,10aS,11S,12S,12aR)-11-(((2-((((6-bromo-7-(methoxymethoxy)-2-oxo-2H-chromen-4-yl)methoxy)carbonyl)(methyl)amino)ethyl)carbamoyl)oxy)-6-(dimethylamino)-2,4a1,10,10,12a-pentamethyl-4-oxo-2-vinyldecahydro-2H,8H-pyrano[3′,2′:1,2]naphtho[1,8-de][1,3]dioxin-12-yl acetate 8 according to the formula

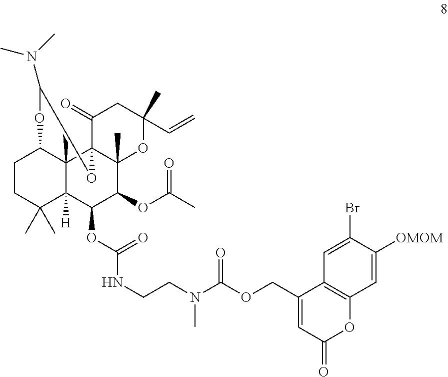

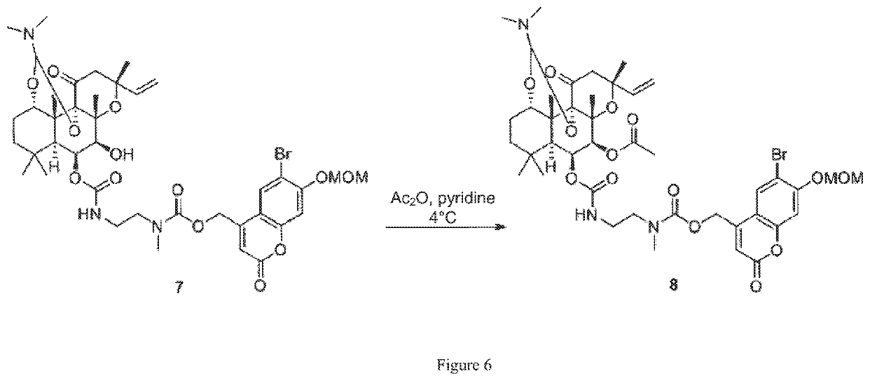

by acetylation of (6-bromo-7-(methoxymethoxy)-2-oxo-2H-chromen-4-yl)methyl (2-(((((2R,4aR,4a1R,6S,10aS,11S,12S,12aR)-6-(dimethylamino)-12-hydroxy-2,4a1,10,10,12a-pentamethyl-4-oxo-2-vinyldecahydro-2H,8H-pyrano[3′,2′:1,2]naphtho[1,8-de][1,3]dioxin-11-yl)oxy)carbonyl)amino)ethyl)(methyl)carbamate 7 (FIG. 6),
e. synthesizing (3R,4aR,5S,6S,6aS,10S,10aR,10bS)-6-(((2-((((6-bromo-7-(methoxymethoxy)-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-yl acetate 9 according to the formula

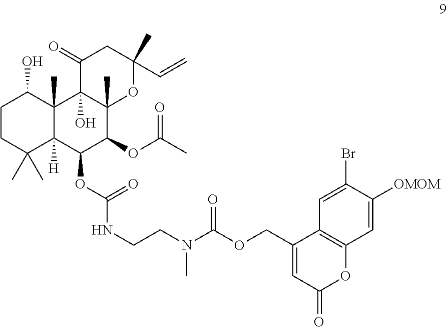

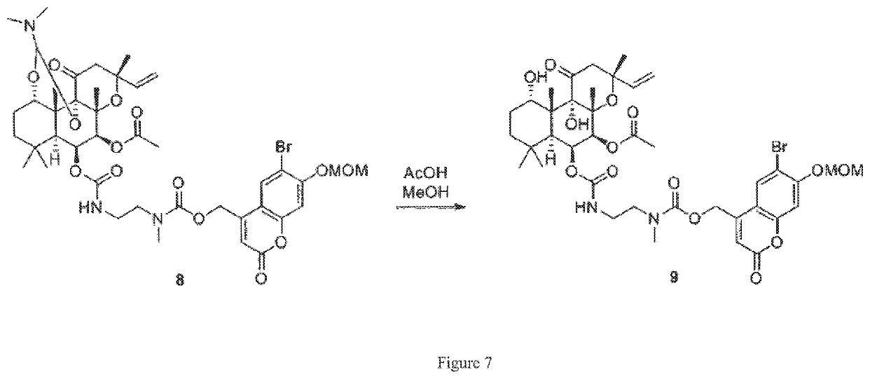

by a second deprotection of (2R,4aR,4a1R,6S,10aS,11S,12S,12aR)-11-(((2-((((6-bromo-7-(methoxymethoxy)-2-oxo-2H-chromen-4-yl)methoxy)carbonyl)(methyl)amino)ethyl)carbamoyl)oxy)-6-(dimethylamino)-2,4a1,10,10,12a-pentamethyl-4-oxo-2-vinyldecahydro-2H,8H-pyrano[3′,2′:1,2]naphtho[1,8-de][1,3]dioxin-12-yl acetate 8 (FIG. 7),
f. synthesizing (3R,4aR,5S,6S,6aS,10S,10aR,10bS)-6-(((2-((((6-bromo-7-hydroxy-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-ylacetate (JCF 1) according to the formula

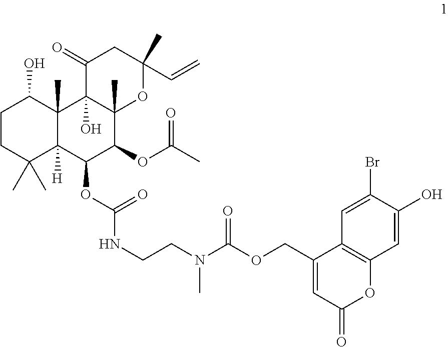

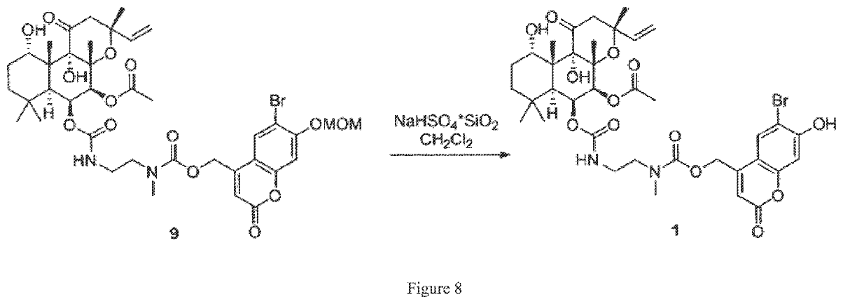

by a third deprotection of (3R,4aR,5S,6S,6aS,10S,10aR,10bS)-6-(((2-((((6-bromo-7-(methoxymethoxy)-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-ylacetate 9 (FIG. 8).

The other excipients of steps a. to f. can advantageously correspond to those of the exemplary embodiment.

In one embodiment of the invention, the first carbamoylation according to step a (FIG. 3) can be carried out with a polar aprotic solvent, for example dimethylformamide (DMF), N-methylformamide (NMF), tetrahydrofuran (THF), dimethyl sulfoxide (DMSO), acetonitrile (ACN), dimethylpropyleneurea (DMPU), pyridine, acetone between approximately 10° C. and 30° C.

In a further embodiment of the invention, the first deprotection according to step b. (FIG. 4) can be carried out with a mineral base and an aqueous alkanolic solvent between approximately 10° C. and 30° C.

The second carbamoylation according to step c. (FIG. 5) can advantageously be carried out with a polar aprotic solvent, e.g., dimethylformamide (DMF), N-methylformamide (NMF), tetrahydrofuran (THF), dimethyl sulfoxide (DMSO), acetonitrile (ACN), dimethylpropyleneurea (DMPU), pyridine, acetone, and an auxiliary base, e.g., diazabicycloundecene (DBU), triethylamine (Et3N), diisopropylethylamine (DIEA), N-methylmorpholine (NMM), tributylamine, and, for example, a catalyst, e.g., py*HCl, with the exclusion of light between approximately 0° C. and 10° C.

The acetylation according to step d. (FIG. 6) can be carried out with a polar aprotic solvent, e.g., dimethylformamide (DMF), N-methylformamide (NMF), tetrahydrofuran (THF), dimethyl sulfoxide (DMSO), acetonitrile (ACN), dimethylpropyleneurea (DMPU), pyridine, acetone, and an acetylation reagent, e.g., acetyl chloride, acetic anhydride, and with the exclusion of light between approximately 0° C. and 10° C.

The second deprotection according to step e. (FIG. 7) can be carried out with an organic acid, for example formic acid, acetic acid, propionic acid, and so on, and any alkanol, for example methanol, ethanol, and so on, between approximately 10° C. and 30° C.

The third deprotection according to step f. (FIG. 8) can be carried out, for example, with a mild catalyst, such as NaHSO₄*SiO₂ in a non-polar aprotic solvent, e.g., CH₂Cl₂, benzene, ether, and so on and between approximately 10° C. and 30° C.

According to an embodiment of the invention, the coumarin-caged forskolin derivative (3R,4aR,5S,6S,6aS,10S,10aR,10bS)-6-(((2-((((6-bromo-7-hydroxy-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-yl acetate (JCF 1) according to the formula:

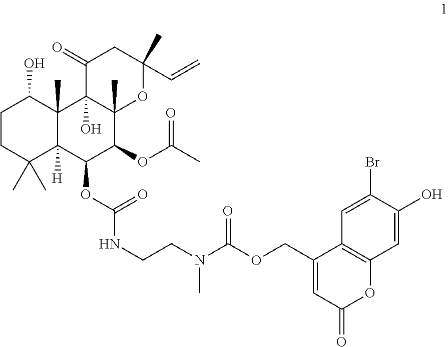

is claimed.

The following intermediates are advantageously provided for the first time during the process according to embodiments of the invention:

(6-bromo-7-(methoxymethoxy)-2-oxo-2H-chromen-4-yl)methyl (2-aminoethyl)(methyl)carbamate 3 according to the formula

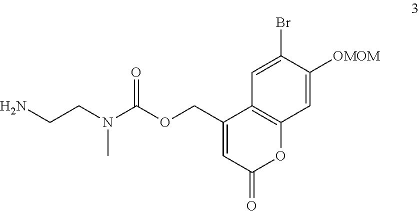

and (6-bromo-7-(methoxymethoxy)-2-oxo-2H-chromen-4-yl)methyl methyl(2-(2,2,2-trifluoroacetamido)ethyl)carbamate 4 according to the formula

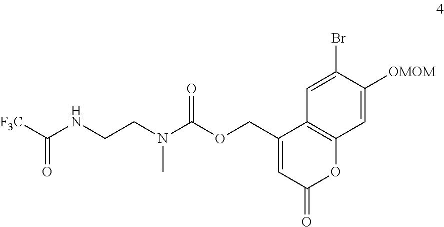

and (6-bromo-7-(methoxymethoxy)-2-oxo-2H-chromen-4-yl)methyl (2-(((((2R,4aR,4a1R,6S,10aS,11S,12S,12aR)-6-(dimethylamino)-12-hydroxy-2,4a1,10,10,12a-pentamethyl-4-oxo-2-vinyldecahydro-2H,8H-pyrano[3′,2′:1,2]naphtho[1,8-de][1,3]dioxin-11-yl)oxy)carbonyl)amino)ethyl)(methyl)carbamate 7 according to the formula

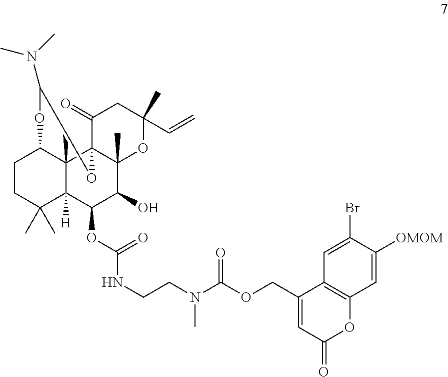

and also (2R,4aR,4a1R,6S,10aS,11S,12S,12aR)-11-(((2-((((6-bromo-7-(methoxymethoxy)-2-oxo-2H-chromen-4-yl)methoxy)carbonyl)(methyl)amino)ethyl)carbamoyl)oxy)-6-(dimethylamino)-2,4a1,10,10,12a-pentamethyl-4-oxo-2-vinyldecahydro-2H,8H-pyrano[3′,2′:1,2]naphtho[1,8-de][1,3]dioxin-12-yl acetate 8 according to the formula

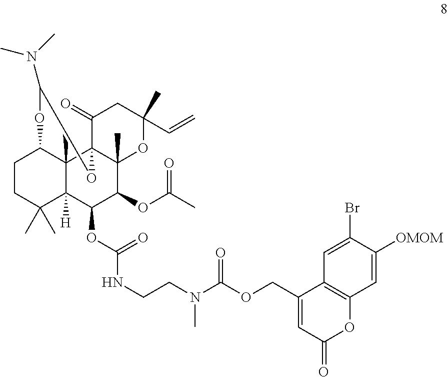

and also (3R,4aR,5S,6S,6aS,10S,10aR,10bS)-6-(((2-((((6-bromo-7-(methoxymethoxy)-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-yl acetate 9 according to the formula

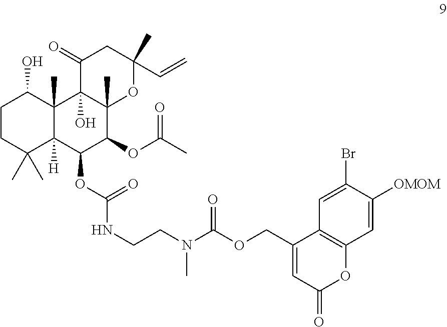

### General Synthesis Description

According to an embodiment of the invention, caged forskolin derivatives for photolysis in cells and tissues with a resulting increase in the cAMP concentration are synthesized in six steps a. to f.

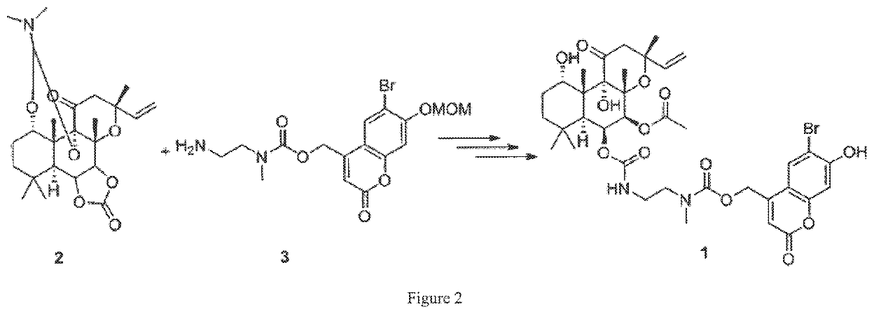

Prior to the coupling of the protected forskolin 2 (FIG. 2 and FIG. 5) to the coumarin cage 3 or its analogs (see FIG. 5), a first carbamoylation of a (pseudo-)halogen-substituted protected coumarin is carried out (analogously to FIG. 3) in order to insert an N-methylalkylenediamine function there. For this purpose, a nitrophenyl carbonate of the protected (pseudo-)halogen-substituted 4-hydroxymethyl coumarin 6 or its analogs is reacted with trifluoro-N-(2-(methylamino)alkyl)acetamide 5 or its analogs. The trifluoroacetyl group is subsequently cleaved off (first deprotection, analogous to FIG. 4).

The amine 3 formed in the process (or its analogs) is coupled in the next step to the completely protected forskolin carbonate 2 by a second carbamoylation (analogously to FIG. 5). The coupled product 7 (or its analogs) is acetylated to the forskolin (acetylation analogous to FIG. 6) and is completely deprotected in two further steps to form the caged forskolin JCF 1 or its analogs (second and third deprotection, analogous to FIGS. 7 and 8).

By means of the aforementioned process steps according to embodiments of the invention, the coumarin-caged forskolin derivatives according to embodiments of the invention according to the general formula

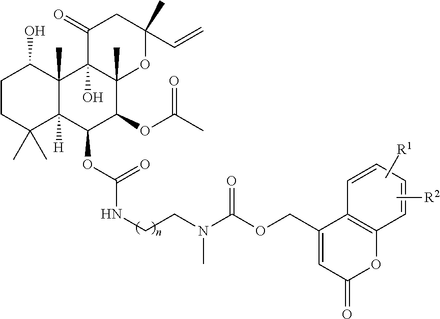

n=1-5; R¹=OH; R²=F, Cl, Br, I, CN, —N3, —OCN, —NCO, —CNO, —SCN, —NCS, —SeCN
are provided particularly advantageously. These are suitable for the use according to further embodiments of the invention.

It is self-evident that for this purpose the educts for the preparation of JCF 1 analogs as described must be adapted accordingly except for the completely protected forskolin 2.

For this purpose, in an embodiment, instead of the specific educt 4 of formula

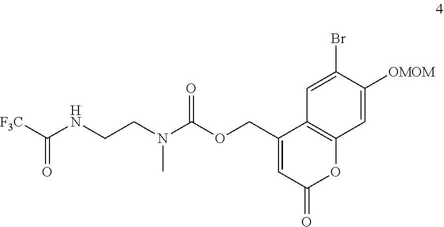

the general educt (pseudo)halogen-(methoxymethoxy-2-oxo-2H-chromen-4-yl)methyl methyl(2-(2,2,2-trifluoroacetamido)alkyl)carbamate of formula

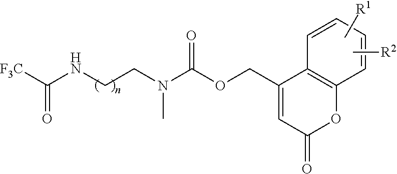

n=1-5; R¹=OH; R²=F, Cl, Br, I, CN, —N₃, —OCN, —NCO, —CNO, —SCN, —NCS, —SeCN
is synthesized in step a. (analogous to FIG. 3) of claim 1 from 2,2,2-trifluoro-N-(2-methylamino alkyl)-acetamide and (pseudohalogen)-methoxymethoxy coumarin-4-ylmethyl 4′-nitrophenyl carbonate to provide the coumarin-caged forskolin derivatives according to the general formula.

Step a. of claim 1 is then as follows for the general synthesis process:

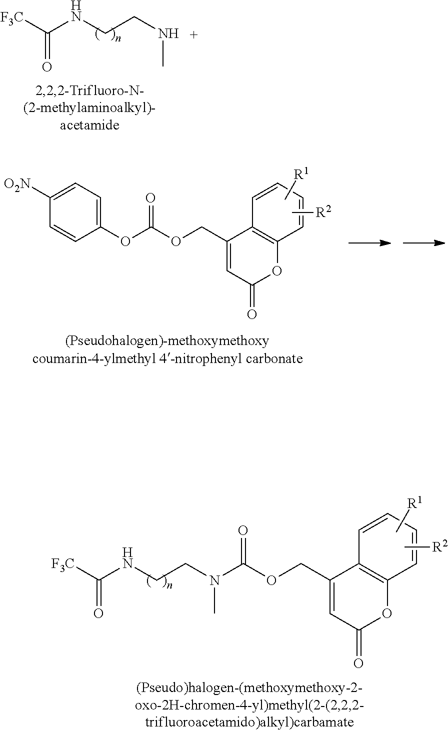

n=1-5; R¹=OH; R²=F, Cl, Br, I, CN, —N₃, —OCN, —NCO, —CNO, —SCN, —NCS, —SeCN

With regard to the process parameters, the further synthesis route of these derivatives corresponds to that of the process for preparing JCF 1.

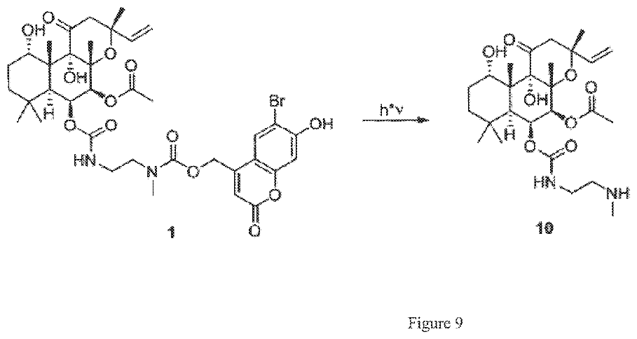

JCF 1 (or its analogs provided according to the invention) is cleaved after irradiation with light in a photolysis process to form forskolin-(2-(methylamino)ethyl)carbamate 10 (or its homologs), CO₂ and the corresponding methylcoumarin derivative (FIG. 9). The remaining coumarin-caged forskolin derivatives according to the invention are cleaved analogously to form the homologous forskolin carbamates, CO₂ and the corresponding methylcoumarin derivatives.

This advantageously allows the use of the coumarin-caged forskolin derivatives according to embodiments of the invention to increase the cAMP concentration in all cell- and tissue-based samples.

For this purpose, the coumarin-caged forskolin derivative can advantageously be introduced into a cell, and, after irradiation, the increase in the intracellular cAMP concentration, which results from the binding of the biologically active forskolin carbamate 10 to peripheral membrane adenylyl cyclases which are endogenously present in the cells, is measured with a fluorescence-based sensitive detection process.

### Exemplary Embodiment

Embodiments of the invention are explained in more detail below with reference to a synthesis route for the coumarin-caged forskolin derivative 1 and the accompanying figures, without this being intended to limit the invention.

The following is shown:

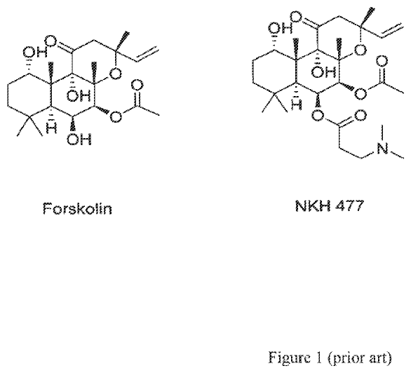

FIG. 1 shows forskolin and NKH 477.

JCF 1 as coumarin-caged forskolin derivative is synthesized in a 6-step synthesis starting from protected forskolin 2 and a coumarin derivative 3 functionalized with N-methylethylenediamine (FIG. 2). The six synthesis steps (FIG. 3 to FIG. 8) and the synthesis products with reference signs 1, 3, 4, 7, 8 and 9 mentioned therein are not previously known from the literature. Their total yield is 5% relative to the carbonate 6.

### Synthesis

(6-Bromo-7-(methoxymethoxy)-2-oxo-2H-chromen-4-yl)methyl methyl(2-(2,2,2-trifluoroacetamido)ethyl)carbamate 4 (FIG. 3).

2,2,2-Trifluoro-N-(2-methylamino-ethyl)-acetamidel^{10,11} 5 (536 mg, 3.15 mmol) and 6-bromo-7-methoxymethoxy coumarin-4-ylmethyl 4′-nitrophenyl carbonate¹² 6 (1000 mg, 2.09 mmol) are stirred at room temperature (RD for 16 h in 15 mL DMF. The solvent is removed on the rotary evaporator under reduced pressure. The crude product is purified by column chromatography (eluent EE:nHex=7:3).

This results in (6-bromo-7-(methoxymethoxy)-2-oxo-2H-chromen-4-yl)methyl methyl(2-(2,2,2-trifluoroacetamido)ethyl)carbamate 4 (850 mg, 1.67 mmol, 80%) as colorless crystals.

MS (ESI+) m/z: [M]⁺ theor. 511.0; exp. 510.9.
(6-Bromo-7-(methoxymethoxy)-2-oxo-2H-chromen-4-methyl (2-aminoethyl)methyl)carbamate 3 (FIG. 4).

Trifluorocarbamate 4 (200 mg, 0.39 mmol) is treated with 25 mL MeOH and 25 mL NaOH_{aq} (0.1 mol/L) in an ultrasonic bath at room temperature for 2 h. The solution is neutralized with HCl_{aq} (0.1 mol/L) and then extracted with CH₂Cl₂ (3×50 mL). The combined organic phases are concentrated on the rotary evaporator under reduced pressure. The crude product is purified using column chromatography (eluent CH₂Cl₂:MeOH=9:1).

This results in (6-bromo-7-(methoxymethoxy)-2-oxo-2H-chromen-4-yl)methyl (2-aminoethyl)(methyl)carbamate 3 (91 mg, 0.22 mmol, 58%) as colorless crystals.

MS (ESI+) m/z: [M]⁺ theor. 417.0; exp. 417.0.
(6-Bromo-7-(methoxymethoxy)-2-oxo-2H-chromen-4-yl)methyl (2-(((((2R,4aR,4a1R,6S,10aS,11S,12S,12aR)-6-(dimethylamino)-12-hydroxy-2,4a1,10,10,12a-pentamethyl-4-oxo-2-vinyldecahydro-2H,8H-pyranol[3′,2′:1,2]naphthol[1,8-de][1,3]dioxin-11-Yl)oxy)carbonyl)amino)ethyl)(methyl)carbamate 7 (FIG. 5)

Pyridine hydrochloride (8.4 mg, 0.073 mmol) and then diazabicycloundecene (DBU, 1.386 mL, 8.9 μmol) are added dropwise to a solution of 7-deacetyl forskolin-6,7-carbonate 1,9-dimethylformamide dimethyl acetal 2 (148 mg, 0.33 mmol) and carbamate 3 (296 mg, 0.72 mmol) in 8 mL pyridine at 0° C. The reaction solution is stirred at 4° C. with the exclusion of daylight in the refrigerator for 5 days. 50 mL CH₂Cl₂ and 10 mL HCl_{aq} (0.1 mol/L) are added. The organic phase is separated out and concentrated on the rotary evaporator under reduced pressure. The crude product is purified using column chromatography (eluent CH₂Cl₂:MeOH=9:1).

This results in (6-bromo-7-(methoxymethoxy)-2-oxo-2H-chromen-4-yl)methyl (2-(((((2R,4aR,4a1R,6S,10aS,11S,12S,12aR)-6-(dimethylamino)-12-hydroxy-2,4a1,10,10,12a-pentamethyl-4-oxo-2-vinyldecahydro-2H,8H-pyrano[3′,2′:1,2]naphtho[1,8-de][1,3]dioxin-11-yl)oxy)carbonyl)amino)ethyl)(methyl)carbamate 7 (250 mg, 0.29 mmol, 88%) as colorless crystals.

MS (ESI+) m/z: [M]⁺ theor. 866.3; exp. 866.3.
(2R,4aR,4a1R,6S,10aS,11S,12S,12aR)-11-(((2-((((6-bromo-7-(methoxymethoxy)-2-oxo-2H-chromen-4-yl)methoxy)carbonyl)(methyl)amino)ethyl)carbamoyl)oxy)-6-(dimethylamino)-2,4a1,10,10,12a-pentamethyl-4-oxo-2-vinyldecahydro-2H,8H-pyrano[3′,2′:1,2]naphtho[1,8-de][1,3]dioxin-12-yl acetate 8 (FIG. 6)

A solution of the alcohol 7 (150 mg, 170 μmol) in 1.5 mL pyridine and 1.5 mL acetic anhydride is stirred at 4° C. excluding daylight in the refrigerator for 2 days. After the addition of 20 mL H₂O, extraction is carried out using CH₂Cl₂ (3×20 mL). The combined organic phases are washed with saturated NaCl solution and dried with Na₂SO₄. The organic phase is separated out and concentrated on the rotary evaporator under reduced pressure. The crude product is purified using column chromatography (eluent CH₂Cl₂:MeOH=9:1).

This results in (2R,4aR,4a1R,6S,10aS,11S,12S,12aR)-11-(((2-((((6-bromo-7-(methoxymethoxy)-2-oxo-2H-chromen-4-yl)methoxy)carbonyl)(methyl)amino)ethyl)carbamoyl)oxy)-6-(dimethylamino)-2,4a1,10,10,12a-pentamethyl-4-oxo-2-vinyldecahydro-2H,8H-pyrano[3′,2′:1,2]naphtho[1,8-de][1,3]dioxin-12-yl acetate 8 (61 mg, 67.4 μmol, 40%) as a colorless foam.

MS (ESI+) m/z: [M]⁺ theor. 908.3; exp. 908.3.
(3R,4aR,5S,6S,6aS,10S,10aR,10bS)-6-(((2-((((6-bromo-7-(methoxymethoxy)-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-yl acetate 9 (FIG. 7).

A solution of the acetal 8 (208 mg, 0.23 mmol) in 3.6 mL MeOH and 2.4 mL glacial acetic acid is stirred overnight at room temperature. 5 mL saturated Na₂CO₃ solution are added, and the aqueous phase is extracted with CH₂Cl₂ (3×10 mL). The combined organic phases are concentrated on the rotary evaporator under reduced pressure. The crude product is purified using column chromatography (eluent CHCl₃:EE=1:1).

This results in (3R,4aR,5S,6S,6aS,10S,10aR,10bS)-6-(((2-(((((6-bromo-7-(methoxymethoxy)-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-yl acetate 9 (95 mg, 0.11 mmol, 48%) as colorless crystals.

MS (ESI+) m/z: [M]⁺ theor. 853.3; exp. 853.2.
(3R,4aR,5S,6S,6aS,10S,10aR,10bS)-6-(((2((((6-bromo-7-hydroxy-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-ylacetate (JCF 1, FIG. 8)

Activated, hot NaHSO₄*SiO₂¹³ (100 mg, 0.56 mmol) is added to a solution of the MOM ether 9 (45 mg, 55 μmol) in 5 mL CH₂Cl₂. The catalyst is activated at 120° C. for 48 hours prior to use. The mixture is stirred at room temperature for 16 h and subsequently filtered. The filtrate is washed with CH₂Cl₂ (2×5 mL). The combined organic phases are concentrated on the rotary evaporator under reduced pressure. The crude product is purified using column chromatography (eluent CHCl₃:EE=1:1).

This results in (3R,4aR,5S,6S,6aS,10S,10aR,10bS)-6-(((2-((((6-bromo-7-hydroxy-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-yl acetate (JCF 1) (29 mg, 36 μmol, 66%) as colorless crystals.

MS (ESI+) m/z: [M+H]⁺ theor. 809.2; exp. 809.2.

### Photolysis

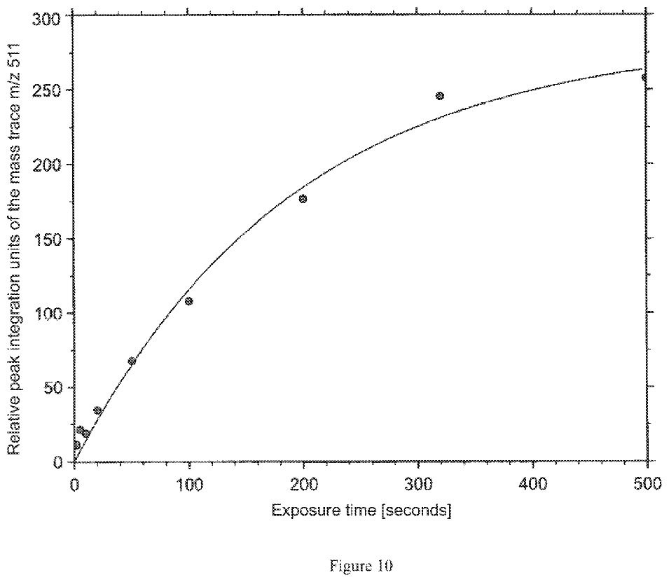

JCF 1 (or its analogs) can be cleaved to form the desired forskolin carbamate 10 under irradiation with light (FIG. 9 and FIG. 10) and thus qualifies for the aforementioned biological use.

The photolysis of JCF 1 is carried out under controlled conditions in order to quantitatively detect the release of forskolin carbamate 10 as a function of the amount of light absorbed: 0.16 ml of a 100 μM solution of JCF 1 in MeOH is added to a quartz glass cuvette (width: 4 mm; depth (optical path): 10 mm->fill level 4 mm). The “Intensilight” light source (excitation lamp of a Nikon TI Eclipse fluorescence microscope) is used as the excitation source, the light of which is guided through a gel light guide (active diameter: 4 mm) and a narrowband bandpass filter (368.8 nm±5 nm) onto the liquid column in the cuvette. 1.58 mW was selected as the excitation power (position 32 (32 times attenuation) on the control unit of the excitation lamp). This results in an irradiance of approximately 12.5 mW/cm². Irradiation was carried out at time intervals of 1 to 500 seconds by manually opening and closing the closure.

The irradiated samples are then examined using mass spectrometry (mass spectrometer: MSQ Plus from ThermoScientific; ionization: ESI-interface with a cone voltage of 50 V, eluent: methanol, water, glacial acetic acid, /50, 50, 0.02/vol, vol, vol; flow rate 0.2 ml/min; direct injections of 20 μl of the respective irradiated samples via a Rheodyne injection valve (7725i)). The mass trace m/z 511 and the mass range m/z 807-811 are recorded. The integrals of the peaks of the chromatogram of the mass trace m/z 511 (see FIG. 10) are evaluated. With an exposure time of 320 seconds, no more signals can be detected in the mass range m/z 807-811 (starting compound), and therefore a complete conversion can be assumed after this time under the above-described conditions.

The reaction can also be carried out analogously with the analogs of JCF-1 prepared according to further embodiments of the invention.

### Validation

The substance JCF 1 described according to embodiments of the invention was validated on eukaryotic cell cultures in which the increase in the intracellular cAMP concentration, which results from the binding of the biologically active forskolin carbamate 10 (FIG. 9) to peripheral membrane adenylyl cyclases which are endogenously present in the cells, using a fluorescence-based sensitive detection process.¹⁴ In this regard, a cell line was used in which a cyclic-nucleotide-gated (CNG) ion channel is constitutively expressed. These channels are typically expressed in olfactory sensory neurons of the olfactory epithelium and open when the intracellular cAMP concentration increases. Cations, inter alia Ca²⁺ ions, flow through the channel into the cell. The inflow of Ca²⁺ can be detected with Ca²⁺-sensitive dyes or genetically encoded Ca²⁺ indicators, such as GCaMP sensors, in a fluorescence reader or with a fluorescence microscope. In order to detect the Ca²⁺ signals, a cell line was prepared which, in addition to the above-mentioned CNG channel additionally constitutively expresses the genetically encoded Ca²⁺ indicator GCaMP3.0 (Tian et al., 2009).¹⁵ The GCaMP3.0 protein consists of a circularly permuted EGFP (enhanced green fluorescent protein) to which a binding peptide for calmodulin consisting of myosin light-chain kinase (M13 peptide) is fused N-terminally and a calmodulin is fused C-terminally. At low intracellular Ca²⁺ concentrations, GCaMP3.0 does not emit fluorescence. As the intracellular Ca²⁺ concentration increases, Ca²⁺ ions bind to the calmodulin. This then interacts with the M13 peptide, and a conformational change of the whole protein results. In this form, GCaMP3.0 fluoresces at 510 nm after irradiation with a wavelength of 480 nm. The Ca²⁺-dependent change in the fluorescence change can be detected and quantified with the aforementioned processes.

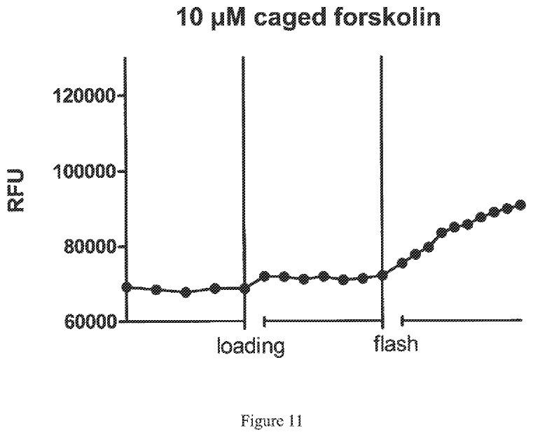

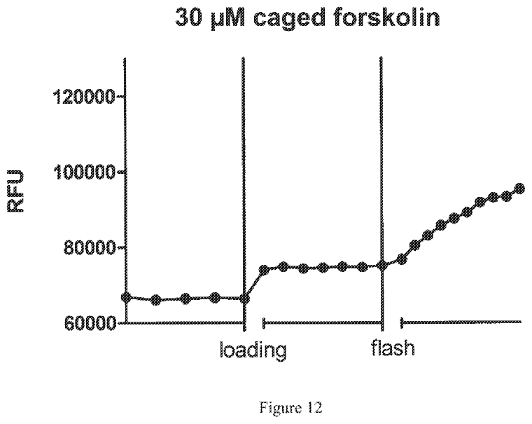

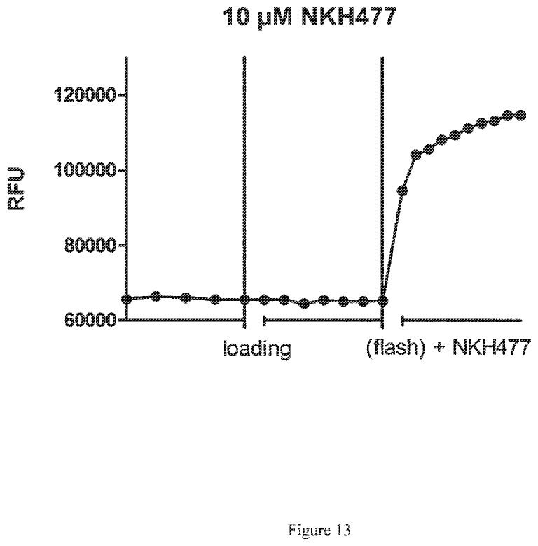

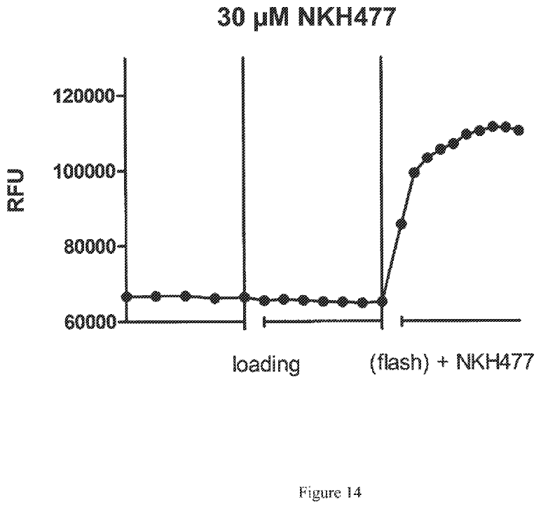

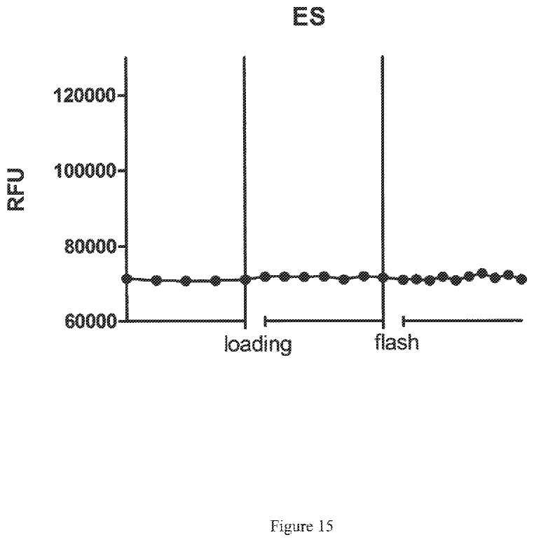

Cells of the cell line described were seeded in 96-well multi-well plates (MWP) and increased to a density of approximately 25,000. The medium was removed and exchanged with extracellular solution containing 100 μM IBMX (isobutylmethylxanthine) for the inhibition of cell-endogenous phosphodiesterases. The basal fluorescence in the wells of 96-well MWP was then measured with a fluorescence reader. Cells in four wells were loaded with JCF 1 (10 μM and 30 μM) for 30 min at room temperature in the dark. The basal fluorescence in the wells was then measured again before the entire 96-well MWP plate was exposed to light using a UV lamp device (FIG. 11, FIG. 12). After the exposure, 10 μM and 30 μM NKH 477 were each pipetted into four independent wells, and the fluorescence changes in the fluorescence reader were measured (FIG. 13, FIG. 14). Cells that had been incubated only with the extracellular solution (with IBMX) were used as negative control (FIG. 15). It was found that the release of forskolin carbamate 10 from the cage compound JCF-1 by UV exposure results in a significant increase in the Ca²⁺-dependent fluorescence emission. Under these conditions, the change in fluorescence reached approximately 50% of the values which were achieved by adding the non-caged NKH 477. No change in Ca²⁺-dependent fluorescence emission was measured in cells that had not been incubated with JCF-1 or with NKH 477 (without cage group).

The use of JCF 1 is universally suitable for all cell- and tissue-based samples in which the intracellular cAMP concentration is to be increased. The possibility of activating the biologically active compound at defined points in time and within the cell by, for example, local release by means of punctiform exposure to light has great advantages over conventional strategies in which an increase in the intracellular cAMP concentration takes place, e.g., via GPCR signaling pathways, via the stimulation of adenylyl cyclases using, for example, NKH 477, or the inhibition of cell-endogenous phosphodiesterases, which hydrolyze the cAMP to AMP, e.g., via IBMX. In the latter processes, changes in the cAMP concentration always occur in the entire cell or within the cell group or tissue group. Moreover, the use of the biologically inactive compound 1 allows the kinetics of cellular processes controlled by increasing the intracellular cAMP concentration to be detected with high time resolution in the sub-second range.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LITERATURE

¹ Ellis-Davies G C R.; Nat. Methods 2007, 4, 619-628.
² Adams S R., Tsien R Y.; Annu. Rev. Physiol. 1993, 55, 755-784.
³ Chaulk S G., MacMillan A M.; Nucleic Acids Res. 1998, 26, 3173-3178.
⁴ Takeuchi K., Takehara K., Kato S., Yagi K.; Am. J. Physiol. 1997, 272, 646-653.
⁵ Kamenetsky M., Middelhaufe S., Bank E M., Levin L R., Buck J., Steegborn C.; J. Mol. Biol. 2006, 362, 623-639.
⁶ Mori M., Takeuchi M., Takaoka H., Hata K., Hayashi Y., Yokoyama M.; J. Cardiovasc. Pharmacol. 1994, 24, 310-316.
⁷ Laurenza A., Khandelwal Y., De Souza N J., Rupp R H., Metzger H., Seamon K B.; Mol. Pharmacol. 1987, 32, 133-139.
⁸ Lai B., Gangopadhyay A K., Gidwani R M., Rajagopalan R., Ghate A V.; Indian J. Chem. 2006, 458, 232-246.
⁹ Hartzell H C., Budnitz D., Mol. Pharmacol. 1992, 41, 880-888.
¹⁰ PHARMACOFORE, INC; Jenkins T E., Seroogy J D., Wray J W.; Tong C C.; WO 2011/133178.
¹¹ Shawe T T., Hepler L P., Chong A M., Fulmer J N., Hansen D B., Watkims J L; Synth. Commun. 1996, 26, 3633-3636.
¹² Fomina N., McFearin C L., Sermsakdi M., Morachis J M., Almutairi A.; Macromolecules 2011, 44, 8590-8597.
¹³ Ramesh C., Ravindranath N., Das B.; J. Org. Chem. 2003, 68, 7101-7103.
¹⁴ Balfanz S., Ehling P., Wachten S., Jordan N., Erber J., Mujagic S. and Baumam, A.; Insect Biochem. Mol. Biol. 2012, 42, 435-445.
¹⁵ Tian L., Hires S. A., Mao T., Huber D., Chiappe M. E., Chalasani S. H., Petreanu L., Akerboom J., McKimey S. A., Schreiter E. R., Bargmam C. I., Jayaraman V., Svoboda K., and Looger L. L.; Nat. Meth. 2009, 6, 875-881.

1. A process for preparing a coumarin-caged forskolin derivative (1) of formula 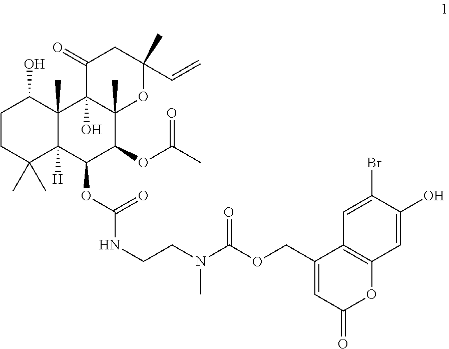
comprising the steps of:
a. synthesizing (6-bromo-7-(methoxymethoxy)-2-oxo-2H-chromen-4-yl)methyl methyl(2-(2,2,2-trifluoroacetamido)ethyl)carbamate (4) of formula 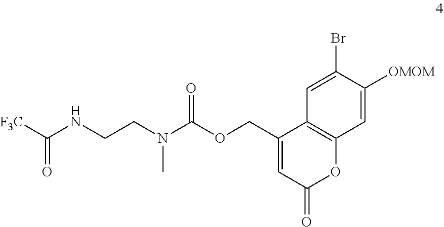
by a first carbamoylation of 2,2,2-trifluoro-N-(2-methylamino-ethyl)-acetamide 5 and 6-bromo-7-methoxymethoxy coumarin-4-ylmethyl 4′-nitrophenyl carbonate 6,
b. synthesizing (6-bromo-7-(methoxymethoxy)-2-oxo-2H-chromen-4-yl)methyl (2-aminoethyl)(methyl)carbamate (3) of formula 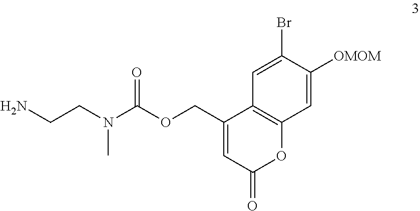
by a first deprotection of (6-bromo-7-(methoxymethoxy)-2-oxo-2H-chromen-4-yl)methyl methyl(2-(2,2,2-trifluoroacetamido)ethyl)carbamate 4,
c. synthesizing (6-bromo-7-(methoxymethoxy)-2-oxo-2H-chromen-4-yl)methyl (2-(((((2R,4aR,4a1R,6S,10aS,11S,12S,12aR)-6-(dimethylamino)-12-hydroxy-2,4a1,10,10,12a-pentamethyl-4-oxo-2-vinyldecahydro-2H,8H-pyrano[3′,2′: 1,2]naphtho[1,8-de][1,3]dioxin-11-yl)oxy)carbonyl)amino)ethyl)(methyl)carbamate (7) of formula 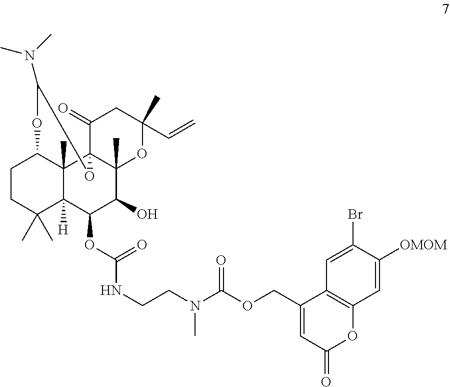
by a second carbamoylation of 7-deacetyl forskolin-6,7-carbonate 1,9-dimethylformamide dimethyl acetal 2 and (6-bromo-7-(methoxymethoxy)-2-oxo-2H-chromen-4-yl)methyl (2-aminoethyl)(methyl)carbamate 3,
d. synthesizing (2R,4aR,4a1R,6S,10aS,11 S,12 S,12aR)-11-(((2-((((6-bromo-7-(methoxymethoxy)-2-oxo-2H-chromen-4-yl)methoxy)carbonyl)(methyl)amino)ethyl)carbamoyl)oxy)-6-(dimethylamino)-2,4a1,10,10,12a-pentamethyl-4-oxo-2-vinyldecahydro-2H, 8H-pyrano[3′,2′:1,2]naphtho[1,8-de][1,3]dioxin-12-yl acetate (8) of formula 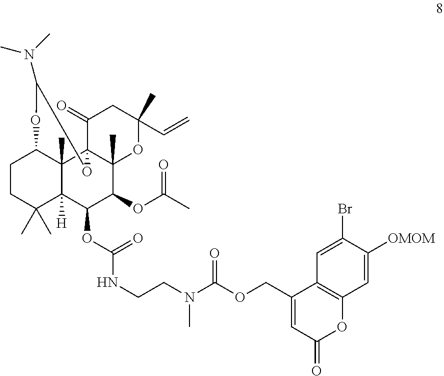
by acetylation of (6-bromo-7-(methoxymethoxy)-2-oxo-2H-chromen-4-yl)methyl (2-(((((2R,4aR,4a1R,6S,10aS,11 S,12S,12aR)-6-(dimethylamino)-12-hydroxy-2,4a1,10,10,12a-pentamethyl-4-oxo-2-vinyldecahydro-2H,8H-pyrano[3′,2′: 1,2]naphtho[1,8-de][1,3]dioxin-11-yl)oxy)carbonyl)amino)ethyl)(methyl)carbamate 7,
e. synthesizing (3R,4aR,5S,6S,6aS,10S,10aR,10bS)-6-(((2-((((6-bromo-7-(methoxymethoxy)-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-yl acetate (9) of formula 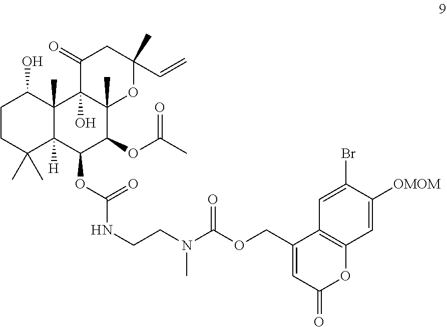
by a second deprotection of (2R,4aR,4a1R,6S,10aS,11S,12S,12aR)-11-(((2-((((6-bromo-7-(methoxymethoxy)-2-oxo-2H-chromen-4-yl)methoxy)carbonyl)(methyl)amino)ethyl)carbamoyl)oxy)-6-(dimethylamino)-2,4a1,10,10,12a-pentamethyl-4-oxo-2-vinyldecahydro-2H,8H-pyrano[3′,2′:1,2]naphtho[1,8-de][1,3]dioxin-12-yl acetate 8,
f. synthesizing (3R,4aR,5 S,6S,6aS,10S,10aR,10bS)-6-(((2-((((6-bromo-7-hydroxy-2-oxo-2H-chromen-4-yl)methoxy)carbonyl)(methyl)amino)ethyl)carbamoyl)oxy)-10,10b-dihydroxy-3 4a,7,7,10a-pentamethyl-1-oxo-3-vinyldodecahydro-1H-benzo[f]chromen-5-yl acetate (1) of formula 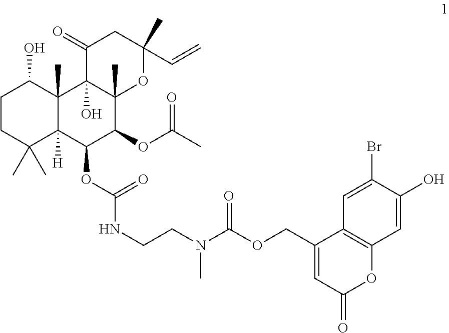
by a third deprotection of (3R,4aR,5S,6S,6aS,10S,10aR,10bS)-6-(((2-((((6-bromo-7-(methoxymethoxy)-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-yl acetate 9.

2. The process according to claim 1,
characterized in that
the first carbamoylation according to step a. is carried out with a polar aprotic solvent between approximately 10° C. and 30° C.

3. The process according to claim 1,
characterized in that
the first deprotection according to step b. is carried out with a base and an aqueous alkanolic solvent between approximately 10° C. and 30° C.

4. The process according to claim 1,
characterized in that
the second carbamoylation according to step c. is carried out with a polar aprotic solvent, a catalyst and an auxiliary base between approximately 0° C. and 10° C.

5. The process according to claim 1,
characterized in that
the acetylation according to step d. is carried out with a polar aprotic solvent and an acetylation reagent between approximately 0° C. and 10° C.

6. The process according to claim 1,
characterized in that
the second deprotection according to step e. is carried out with an organic acid and an alkanol between approximately 10° C. and 30° C.

7. The process according to claim 1,
characterized in that
the third deprotection according to step f. is carried out with a catalyst and with a non-polar aprotic solvent between approximately 10° C. and 30° C.

8. The process according to claim 1,
characterized in that
in step a., instead of (6-bromo-7-(methoxymethoxy)-2-oxo-2H-chromen-4-yl)methyl methyl(2-(2,2,2-trifluoroacetamido)ethyl)carbamate 4 to prepare JCF 1, the general educt (pseudo)halogen-(methoxymethoxy-2-oxo-2H-chromen-4-yl)methyl methyl(2-(2,2,2-trifluoroacetamido)alkyl)carbamate of formula 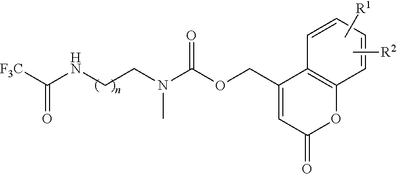
wherein n is=1-5; R¹ is=OH; and R² is=F, Cl, Br, I, CN, —N₃, —OCN, —NCO, —CNO, —SCN, —NCS, or —SeCN,
is synthesized from 2,2,2-trifluoro-N-(2-methylamino alkyl)-acetamide and (pseudohalogen)-methoxymethoxy coumarin-4-ylmethyl 4′-nitrophenyl carbonate to prepare a coumarin-caged forskolin derivative according to the general formula 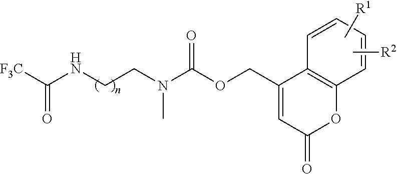
wherein n is=1-5; R¹ is=OH; and R² is=F, Cl, Br, I, CN, —N₃, —OCN, —NCO, —CNO, —SCN, —NCS, or —SeCN.

9. A coumarin-caged forskolin derivative of formula 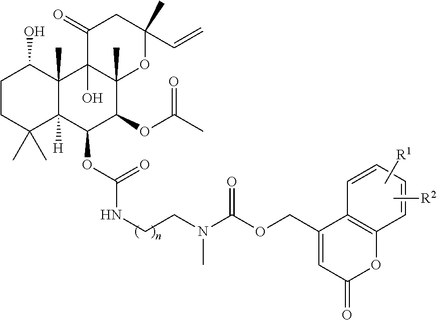
wherein n is=1-5; R¹ is=OH; and R² is=F, Cl, Br, I, CN, —N₃, —OCN, —NCO, —CNO, —SCN, —NCS, or —SeCN.

10. The coumarin-caged forskolin derivative of claim 9, wherein the derivative is (3R,4aR,5S,6S,6aS,10S,10aR,10bS)-6-(((2-((((6-bromo-7-hydroxy-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-yl acetate (1) of formula:

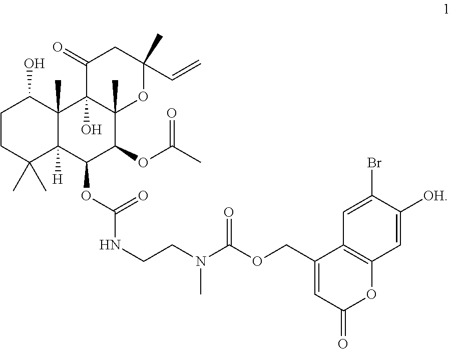

11. 6-Bromo-7-(methoxymethoxy)-2-oxo-2H-chromen-4-yl)methyl (2-aminoethyl)(methyl)carbamate 3 of formula

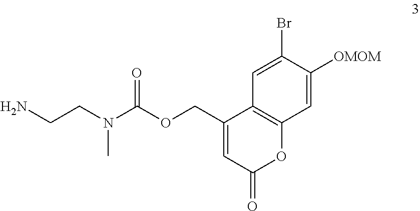

12-14. (canceled)

15. The coumarin-caged forskolin derivative of claim 9, wherein
the derivative is (3R,4aR,5S,6S,6aS,10S,10aR,10bS)-6-(((2-((((6-bromo-7-(methoxymethoxy)-2-oxo-2H-chromen-4-yl)methoxy)carbonyl)(methyl)amino)ethyl)carbamoyl)oxy)-10,10b-dihydroxy-3,4a,7,7,10a-pentamethyl-1-oxo-3-vinyldodecahydro-1H-benzo[f]chromen-5-yl acetate (9) of formula

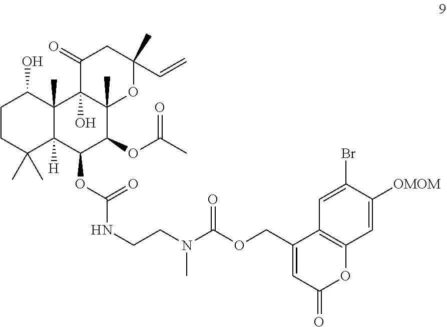

16. A method of irradiating the coumarin-caged forskolin derivative of claim 9 with light in a photolysis process, wherein the derivative is cleaved after irradiation with light in a photolysis process to form forskolin carbamate 10, CO₂ and the corresponding methylcoumarin derivative.

17. A method of increasing cAMP concentration and Ca₂⁺ concentration in cell- and tissue-based samples, comprising introducing the coumarin-caged forskolin derivative of claim 9 into a cell or tissue, and irradiating the coumarin-caged forskolin derivative with light in a photolysis process.

18. The method of claim 16,
characterized in that
the coumarin-caged forskolin derivative is introduced into a cell or tissue prior to irradiation, and, after irradiation, the increase in the intracellular Ca²⁺ concentration resulting from the increase in the cAMP concentration is measured with a fluorescence-based detection process.

19. A method of irradiating the coumarin-caged forskolin derivative of claim 10 with light in a photolysis process, wherein the derivative is cleaved after irradiation with light in a photolysis process to form forskolin carbamate 10, CO₂ and the corresponding methylcoumarin derivative.

20. The method of claim 17,
characterized in that
the increase in the intracellular Ca²⁺ concentration resulting from the increase in the cAMP concentration is measured with a fluorescence-based detection process.
